(19) United States
(12) Patent Application Publication
(10) Pub. No.: US 2025/0380984 A1
(43) Pub. Date: Dec. 18, 2025

(54) LEAFLET REMOVAL DEVICE

(71) Applicant: BOSTON SCIENTIFIC SCIMED, INC., MAPLE GROVE, MN (US)

(72) Inventors: James M. Anderson, Corcoran, MN (US); Eric Michael Petersen, Maple Grove, MN (US)

(73) Assignee: BOSTON SCIENTIFIC SCIMED, INC., MAPLE GROVE, MN (US)

(21) Appl. No.: 19/240,361

(22) Filed: Jun. 17, 2025

Related U.S. Application Data
(60) Provisional application No. 63/661,189, filed on Jun. 18, 2024.

(51) Int. Cl. A61B 18/14; A61B 18/00; A61M 25/10

(52) U.S. Cl. CPC A61B 18/1492; A61M 25/10; A61B 2018/00083; A61B 2018/0022; A61B 2018/00369; A61B 2018/00595; A61B 2018/1407; A61M 2025/1075; A61M 2025/1086; A61M 2205/0238; A61M 2210/125

(57) ABSTRACT

A medical device that is adapted for excising a valve leaflet portion of a valve leaflet from a heart valve includes an elongate shaft including a distal region and an inflatable balloon that is secured to the distal region and that is adapted to extend through the heart valve. The inflatable balloon has an outer surface and includes a deflated configuration and an inflated configuration. An electrocautery electrode is disposed relative to the inflatable balloon such that the electrocautery electrode is positioned to excise the valve leaflet portion when RF energy is applied to the electrocautery electrode and the inflatable balloon is in the inflated configuration. In some cases, the heart valve includes an artificial heart valve. In some cases, the valve leaflet includes an artificial valve leaflet.

### CROSS REFERENCE TO RELATED APPLICATION

This application is a continuation of U.S. Patent Application Ser. No. 63/661,189, filed Jun. 18 2024, entitled “LEAFLET REMOVAL DEVICE”, which is incorporated by reference herein in its entirety.

### TECHNICAL FIELD

The present disclosure relates generally to medical devices. More particularly, the present disclosure pertains to medical devices for excising cardiac valve leaflets.

### BACKGROUND

A wide variety of intracorporeal medical devices have been developed for medical use, for example, intravascular use. Some of these devices include devices for lacerating cardiac valve leaflets. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for excising cardiac valve leaflets. An example may be found in a medical device that is adapted for excising a valve leaflet portion from a heart valve. The medical device includes an elongate shaft including a distal region and an inflatable balloon that is secured to the distal region. The inflatable balloon is adapted to extend through the heart valve. The inflatable balloon includes a deflated configuration and an inflated configuration and has an outer surface. An electrocautery electrode is disposed relative to the inflatable balloon such that the electrocautery electrode is positioned to excise the valve leaflet portion when RF energy is applied to the electrocautery electrode and the inflatable balloon is in the inflated configuration.

Alternatively or additionally, the heart valve may include an artificial heart valve.

Alternatively or additionally, the valve leaflet may include an artificial valve leaflet.

Alternatively or additionally, the outer surface of the inflatable balloon may be adapted to capture the excised valve leaflet portion.

Alternatively or additionally, the medical device may further include a layer disposed on the outer surface of the inflatable balloon that is to adapted capture the excised valve leaflet portion.

Alternatively or additionally, the inflatable balloon may include an inner balloon wall and an outer balloon wall defining a space therebetween, with the outer balloon wall defining the outer surface. The outer balloon wall may include a plurality of pores. A source of vacuum may be fluidly coupled with the space defined between the inner balloon wall and the outer balloon wall.

Alternatively or additionally, the outer surface of the inflatable balloon may be adapted to fold over the excised valve leaflet portion when the inflatable balloon is deflated.

Alternatively or additionally, the electrocautery electrode may be disposed on the outer surface of the inflatable balloon.

Alternatively or additionally, the medical device may further include an insulative layer disposed on the outer surface of the inflatable balloon.

Alternatively or additionally, the medical device may further include an ancillary device that is adapted to extend at least partially around the inflatable balloon.

Alternatively or additionally, the electrocautery electrode may be disposed on the ancillary device.

Alternatively or additionally, the electrocautery electrode may include a mono-polar loop electrode.

Alternatively or additionally, the electrocautery electrode may include a bi-polar loop electrode.

Another example may be found in a medical device that is adapted for excising a portion of an aortic valve leaflet from an aortic valve. The medical device includes an elongate shaft including a distal region. An inflatable balloon is secured to the distal region and is adapted to extend through the aortic valve. The inflatable balloon includes a deflated configuration and an inflated configuration and has an outer surface. An ancillary device is adapted to extend at least partially over the inflatable balloon. An electrocautery electrode is disposed between the inflatable balloon and the ancillary device such that the electrocautery electrode is positioned to excise the portion of the aortic valve leaflet when energy is applied to the electrocautery electrode and the inflatable balloon is in the inflated configuration.

Alternatively or additionally, the aortic valve comprises a previously implanted artificial aortic valve.

Alternatively or additionally, the outer surface of the inflatable balloon may be adapted to capture a portion of the aortic valve leaflet that was excised by the electrocautery electrode.

Another example may be found in a medical device that is adapted for excising material from a heart valve. The medical device includes an elongate shaft including a distal region and an inflatable balloon that is secured to the distal region. The inflatable balloon is adapted to extend through the heart valve. The inflatable balloon includes a deflated configuration and an inflated configuration and includes an outer surface. An electrode is disposed relative to the inflatable balloon such that the electrode is positioned to excise the material when energy is applied to the electrode and the inflatable balloon is in the inflated configuration. The outer surface of the inflatable balloon is adapted to capture the material excised by the electrode.

Alternatively or additionally, the medical device may further include an adhesive layer that is disposed on at least a portion of the outer surface of the inflatable balloon.

Alternatively or additionally, the inflatable balloon may include an inner balloon wall and an outer balloon wall defining a space therebetween, with the outer balloon wall defining the outer surface. The outer balloon wall may include a plurality of pores. A source of vacuum may be fluidly coupled with the space defined between the inner balloon wall and the outer balloon wall.

Alternatively or additionally, the outer surface of the inflatable balloon may be adapted to fold over the excised portion of the valve leaflet when the inflatable balloon is deflated.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cutaway view showing a replacement heart valve implant positioned within a native valve annulus of a heart;

FIG. 2 is a schematic view of an illustrative medical device;

FIG. 3 is a schematic view of a portion of the illustrative medical device of FIG. 2;

FIG. 4 is a schematic view of an illustrative inflatable balloon construction;

FIG. 5 is a schematic view of an illustrative inflatable balloon construction;

FIG. 6 is a schematic view of an illustrative inflatable balloon construction;

FIG. 7 is a schematic view of an illustrative inflatable balloon construction;

FIG. 8 is a schematic view of an illustrative inflatable balloon construction;

FIG. 9 is a schematic view of an illustrative inflatable balloon construction;

FIG. 10 is a schematic view of an illustrative inflatable balloon construction;

FIG. 11 is a schematic view of an illustrative inflatable balloon construction;

FIG. 12 is a schematic view of an illustrative inflatable balloon construction;

FIG. 13 is a schematic view of an illustrative inflatable balloon construction;

FIG. 14 is a schematic view of an illustrative inflatable balloon construction;

FIG. 15 is a schematic view of an illustrative electrode arrangement;

FIG. 16 is a schematic view of an illustrative electrode arrangement;

FIG. 17 is a schematic view of an illustrative medical device;

FIG. 18 is a schematic view of an illustrative medical device; and

FIG. 19 is a schematic view of an illustrative medical device.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

A number of patients receive artificial heart valves for a variety of reasons including valve malfunction due to calcium accumulation. When an artificial heart valve is implanted, the artificial heart valve may have an expandable frame that presses the native valve leaflets away from the native position of the native valve leaflets. In some instances, the native valve is the aortic valve, and the artificial heart valve is an artificial aortic valve. In some instances, it is possible for one or more of the native valve leaflets, when pressed to the side, to at least partially or even completely block an ostium of one of the coronary arteries. Not only does this present possible health concerns for the patient, particularly if an ostium is completely blocked, but even when an ostium is only partially blocked and thus still permits blood flow, this may present difficulties in subsequently being able to perform balloon angioplasty, or place a stent, in one of the coronary arteries. In some instances, it may be beneficial to slice or lacerate with opportunity to remove or excise one or more of the native valve leaflets prior to implantation of the artificial heart valve so that when the native valve leaflets are pressed to the side by the expandable frame of the artificial heart valve, the native valve leaflets do not block an ostium of any of the coronary arteries.

In some instances, a patient may already have an implanted artificial heart valve such as an artificial aortic valve. The artificial valve leaflets forming part of the already implanted artificial heart valve can be just as problematic with respect to potentially blocking a cardiac artery ostium when displaced to the side when a second artificial heart valve is implanted in place of the first artificial heart valve. The artificial valve leaflets forming part of the artificial heart valve may, for example, be made from full porcine root, porcine or bovine pericardium, or may be polymeric. In some instances, artificial valve leaflets may be made of polymers such as Dacron or Gore-Tex. As discussed here, reference to a valve leaflet may refer to either a native valve leaflet or an artificial valve leaflet.

In some instances, a medical device that is adapted for excising a valve leaflet portion of a valve leaflet from a heart valve includes an elongate shaft including a distal region and an inflatable balloon that is secured to the distal region and that is adapted to extend through the heart valve. The inflatable balloon has an outer surface and includes a deflated configuration and an inflated configuration. An electrocautery electrode is disposed relative to the inflatable balloon such that the electrocautery electrode is positioned to excise the valve leaflet portion when RF energy is applied to the electrocautery electrode and the inflatable balloon is in the inflated configuration. In some cases, the heart valve includes an artificial heart valve. In some cases, the valve leaflet includes an artificial valve leaflet.

In some cases, the outer surface of the inflatable balloon may be adapted to capture the excised valve leaflet portion. As an example, the medical device may further include a layer disposed on the outer surface of the inflatable balloon that may capture the excised valve leaflet portion. As another example, the inflatable balloon may include an inner balloon wall and an outer balloon wall defining a space therebetween, where the outer balloon wall defines the outer surface of the inflatable balloon. The outer balloon wall includes a plurality of pores. A source of vacuum may be fluidly coupled with the space defined between the inner balloon wall and the outer balloon wall. In some cases, the outer surface of the inflatable balloon may be adapted to fold over the excised valve leaflet portion when the inflatable balloon is deflated.

In some cases, the electrocautery electrode may be disposed on the outer surface of the inflatable balloon. The medical device may further include an insulative layer that is disposed on the outer surface of the inflatable balloon. In some cases, the medical device may further include an ancillary device that is adapted to extend at least partially around the inflatable balloon. In some cases, the electrocautery electrode may be disposed on the ancillary device. As an example, the electrocautery electrode may include a mono-polar loop electrode. As another example, the electrocautery electrode may include a bi-polar loop electrode.

In some instances, a medical device that is adapted for excising a portion of an aortic valve leaflet from an aortic valve includes an elongate shaft including a distal region and an inflatable balloon that is secured to the distal region and is adapted to extend through the heart valve. The inflatable balloon has an outer surface and includes a deflated configuration and an inflated configuration. An ancillary device is adapted to extend at least partially over the inflatable balloon and an electrocautery electrode is disposed between the inflatable balloon and the ancillary device such that the electrocautery electrode is positioned to excise the portion of the aortic valve leaflet when energy is applied to the electrocautery electrode and the inflatable balloon is in the inflated configuration. In some cases, the aortic valve is a previously implanted artificial aortic valve. In some cases, the outer surface of the inflatable balloon may be adapted to capture a portion of the aortic valve leaflet excised by the electrocautery electrode.

In some instances, a medical device that is adapted for excising material from a heart valve includes an elongate shaft including a distal region and an inflatable balloon that is secured to the distal region and is adapted to extend through the heart valve. The inflatable balloon has an outer surface and includes a deflated configuration and an inflated configuration. An electrode is disposed relative to the inflatable balloon such that the electrode is positioned to excise a portion of the valve leaflet when energy is applied to the electrode and the inflatable balloon is in the inflated configuration. The outer surface of the inflatable balloon is adapted to capture the excised material. The excised material may include native leaflet material. The excised material may include any excess tissue. The excised material may include diseased tissue such as commissural tissue and annulus tissue. The excised material may include artificial sealing materials, for example.

In some cases, the medical device may further include an adhesive layer that is disposed on at least a portion of the outer surface of the inflatable balloon. In some cases, the inflatable balloon includes an inner balloon wall and an outer balloon wall defining a space therebetween and the outer surface of the inflatable balloon. The outer balloon wall includes a plurality of pores. A source of vacuum may be fluidly coupled with the space defined between the inner balloon wall and the outer balloon wall. In some cases, the outer surface of the inflatable balloon may be adapted to fold over the excised portion of the valve leaflet when the inflatable balloon is deflated.

As noted, the medical devices described herein may be used in excising portions of valve leaflets regardless of whether the valve leaflets are native valve leaflets or artificial valve leaflets. In some cases, portions of the native valve leaflets may be excised prior to implantation of an artificial heart valve in order to avoid possible issues with one or more of the native valve leaflets from obscuring an ostium of one of the coronary arteries. Even if blood is able to flow through the ostium and into one of the coronary arteries, having the ostium even partially blocked with a native valve leaflet can potentially cause difficulties with subsequent procedures such as performing angioplasty within one of the coronary arteries or implanting a stent within one of the coronary arteries.

In some cases, a second artificial heart valve may be implanted within a previously implanted artificial heart valve. There may be a desire to excise one or more of the artificial valve leaflets within the previously implanted artificial heart valve before implanting the replacement artificial heart valve within the previously implanted artificial heart valve. In some cases, excising one or more of the artificial valve leaflets may help reduce or eliminate potential issues with the artificial valve leaflets of the previously implanted artificial heart valve interfering with operation of the replacement artificial heart valve and/or potentially blocking an ostium of one of the coronary arteries.

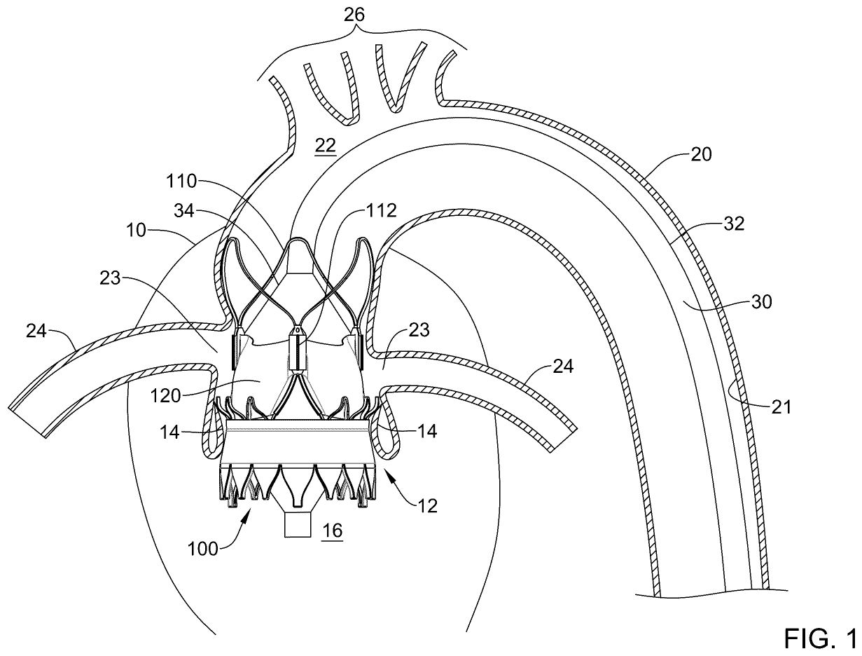

FIG. 1 is a schematic partial cut-away view of a portion of a patient's heart 10 including an aortic valve 12 having native valve leaflets 14 disposed within and/or extending from the native valve annulus, a left ventricle 16, and certain connected vasculature, such as an aorta 20 connected to the aortic valve 12 of the patient's heart 10 by an aortic arch 22 and an ascending aorta, the coronary ostia 23 of the coronary arteries 24, which extend from the aortic sinuses and/or the ascending aorta, and other large arteries 26 (e.g., subclavian and/or carotid arteries, etc.) that extend from the aortic arch 22 to important internal organs. For the purpose of this disclosure, the discussion herein is directed toward treating the aortic valve 12 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

FIG. 1 further illustrates selected aspects of a replacement heart valve implant 100 positioned within the aortic valve 12 and/or the native valve annulus of the aortic valve 12. Some non-limiting examples of the replacement heart valve 100 may include the ACURATE NEO2™, the ACURATE PRIME™, and/or family members thereof from Boston Scientific. It should be appreciated that the replacement heart valve implant 100 can be any type of replacement heart valve (e.g., a mitral valve, an aortic valve, etc.). In use, the replacement heart valve implant 100 may be implanted (e.g., such as through transcatheter delivery) in the aortic valve 12 of the heart 10. The replacement heart valve implant 100 can be configured to allow one-way flow through the replacement heart valve implant 100 from an inflow end to an outflow end.

The replacement heart valve implant 100 may include an expandable framework 110 defining a central lumen. Some suitable but non-limiting examples of materials that may be used to form the expandable framework 110, including but not limited to metals and metal alloys, composites, ceramics, polymers, and the like, are described below. The replacement heart valve implant 100 and/or the expandable framework 110 may be configured to shift between a radially collapsed configuration and a radially expanded configuration. In some instances, the expandable framework 110 may be self-expanding. In some instances, the expandable framework 110 may be self-biased toward the radially expanded configuration. In some cases, the expandable framework 110 may be mechanically expandable. As an example, the expandable framework 110 may be balloon expandable.

In some instances, the replacement heart valve implant 100 may include a plurality of valve leaflets 120 disposed within the central lumen. The plurality of valve leaflets 120 may be coupled, secured, and/or fixedly attached to the expandable framework 110 at a plurality of commissures 112. The plurality of valve leaflets 120 may be configured to shift between an open position and a closed position. The plurality of valve leaflets 120 may be configured to substantially restrict fluid flow through the replacement heart valve implant 100 in the closed position. The plurality of valve leaflets 120 may move apart from each other in the open position to permit fluid flow through the replacement heart valve implant 100.

In some cases, the plurality of valve leaflets 120 may include a polymer such as a thermoplastic polymer. In some cases, the plurality of valve leaflets 120 may include at least 50 percent by weight of a polymer. In some instances, the plurality of valve leaflets 120 may be formed from porcine pericardium, bovine pericardium, or other living tissue. Other configurations and/or materials are also contemplated.

As seen in FIG. 1, a medical device 30 extends through the aortic arch 22 and into an interior of the previously implanted replacement heart valve implant 100. As will be described, the medical device 30 may be used for excising at least part of one or more of the valve leaflets 120 of the replacement heart valve implant 100. The medical device 30 includes an elongate shaft 32 that extends through the aortic arch 22 and in some cases contacts an interior wall 21 of the aorta 20. The medical device 30 includes an inflatable balloon 34. In some cases, contacting the interior wall 21 of the aorta 20 may help in guiding the medical device 30 into position relative to the replacement heart valve implant 100. Once the medical device 30 has been positioned relative to the replacement heart valve implant 100 (or relative to the native valve leaflets 14 if the aortic valve 12 is still intact and no replacement heart valve implant 100 was previously implanted), the medical device 30 may be used to excise at least part of one or more of the valve leaflets 120 (or the valve leaflets 14) by inflating the inflatable balloon 34.

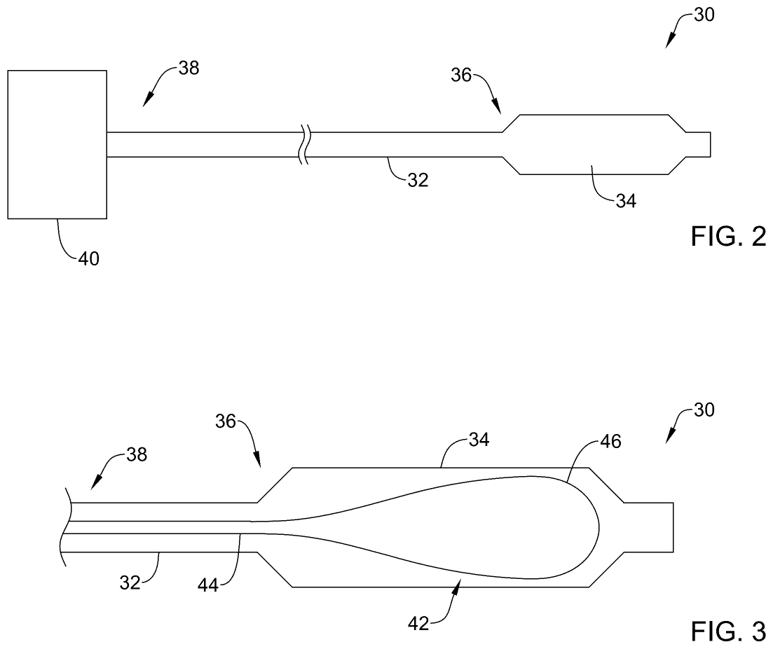

FIG. 2 is a schematic view of the medical device 30. The elongate shaft 32 includes a distal region 36 and extends proximally to a proximal region 38. In some cases, the medical device 30 includes a hub 40 that is secured relative to the proximal region 38 of the elongate shaft 32. While not shown in FIG. 2, the hub 40 may include one or more ports or connections to lumens extending within the elongate shaft 32. The hub 40 may accommodate a guidewire lumen extending through the hub 40 and through the elongate shaft 32. The hub 40 may accommodate an electrical connection to an electrical conductor (not shown) that extends through a lumen within the elongate shaft 32 and that is electrically coupled with an electrocautery electrode 42. The hub 40 may accommodate a fluid coupling lumen for providing an inflation fluid through a lumen extending within the elongate shaft 32 for inflating the inflatable balloon 34.

FIG. 3 is a schematic view of a distal portion of the medical device 30, showing the inflatable balloon 34 and the electrocautery electrode 42. The electrocautery electrode 42 may be used to excise portions of at least one of the valve leaflets 120 (or the valve leaflets 14) by supplying energy such as RF (Radio Frequency) energy to the electrocautery electrode 42. As shown, the electrocautery electrode 42 may be considered as being a loop electrode, with a conductive member 44 extending distally relative to the elongate shaft 32, forming a loop structure 46 relative to the inflatable balloon 34 and then extending proximally relative to the elongate shaft 32. It will be appreciated that this is merely one example of how the electrocautery electrode 42 may be arranged relative to the inflatable balloon 34. In some cases, the electrocautery electrode 42 may be a mono-polar electrode, bi-polar electrode or even a quadripolar electrode, for example.

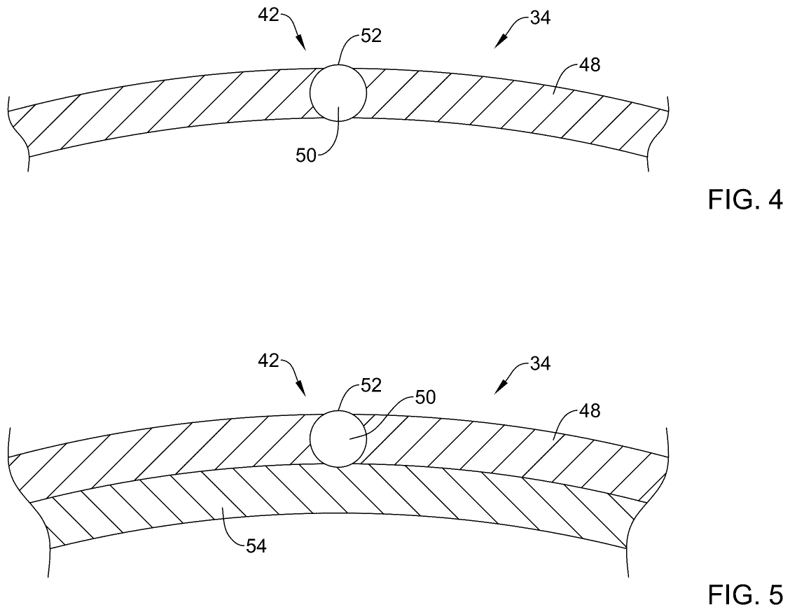

The inflatable balloon 34 may incorporate the electrocautery electrode 42 in a variety of ways. FIGS. 4 through 10 provide illustrative but non-limiting examples of how the electrocautery electrode 42 may be attached to, or incorporated into, the inflatable balloon 34. FIG. 4 provides an example of the inflatable balloon 34 having a single wall construction. As shown, the inflatable balloon 34 includes a wall 48. The electrocautery electrode 42 includes a conductive member 50 that is disposed within the wall 48, and is exposed along an outer edge 52. The wall 48 may be constructed of any desired biocompatible polymer suitable for forming inflatable balloons. The conductive member 50 may be made from any desired electrically conductive material such as a metal. In some cases, the conductive member 50 may be made of gold.

FIG. 5 provides an example of the inflatable balloon 34 having a dual wall construction. As shown, the inflatable balloon 34 includes the wall 48, forming an outer wall, and an inner wall 54. The electrocautery electrode 42 includes the conductive member 50 that is disposed within the wall 48, and is exposed along its outer edge 52. The wall 48 may be constructed of any desired polymer suitable for forming inflatable balloons. The inner wall 54 may be may be constructed of any desired biocompatible polymer suitable for forming inflatable balloons. The conductive member 50 may be made from any desired electrically conductive material such as a metal. In some cases, the conductive member 50 may be made of gold.

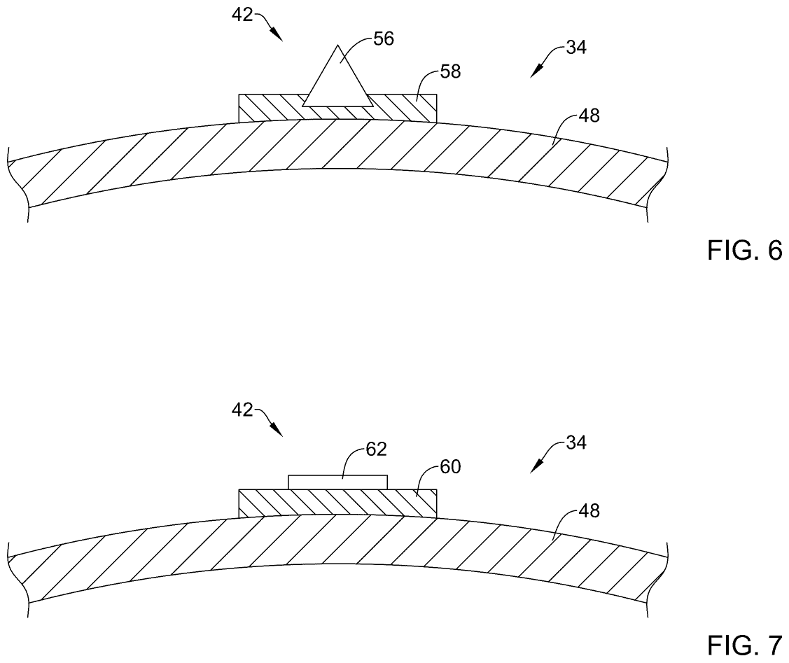

FIG. 6 provides an example of the inflatable balloon 34 having a single wall construction, with a conductive member 56 that is adhesively secured relative to the inflatable balloon 34. As shown, the inflatable balloon 34 includes the wall 48. The electrocautery electrode 42 includes a conductive member 56 that is at least partially embedded within an attachment layer 58. While the conductive member 56 may have a variety of cross-sectional profile, as shown the conductive member 56 has a triangular cross-sectional profile. In some cases, this provides a maximum area of the conductive member 56 for securement to and within the attachment layer 58 and also providing a minimum dimension for the exposed electrode. Providing a minimum dimension for the exposed electrode may help in increasing the relative electrical density that can be provided to the tissue being excised. The conductive member 56 may be made from any desired electrically conductive material such as a metal. In some cases, the conductive member 56 may be made of gold.

In some cases, the attachment layer 58 may be a biocompatible polymeric layer that is adhesively secured to both the conductive member 56 and the wall 48. In some cases, the attachment layer 58 may also be electrically insulative. In some cases, the conductive member 56 may be molded or thermally secured within the attachment layer 58. This may include pre-post molded securement of the conductive member 56. Examples of suitable materials for the attachment layer 56 include polyamides, thermoplastic polyurethane elastomers available under the Pellethane® name, and aromatic polyurethanes available under the Tecothane® name. In some cases, adhesives may also be used to provide additional securement and to function as a strain relief.

FIG. 7 provides another example of the inflatable balloon 34 having a single wall construction, with a conductive member 62 that is adhesively secured relative to the inflatable balloon 34. As shown, the inflatable balloon 34 includes the wall 48. A polymeric layer 60 is disposed on the wall 48. The polymeric layer 60 may be any of a variety of different biocompatible polymers. As an example, the polymeric layer 60 may include a polyimide. The electrocautery electrode 42 includes a conductive member 62 that may be disposed on the polymeric layer 60. The conductive member 62 may have a variety of different cross-sectional profiles. As shown, the conductive member 62 has a rectilinear cross-sectional profile. The conductive member 62 may be made from any desired electrically conductive material such as a metal. In some cases, the conductive member 62 may be made of gold. In some cases, the conductive member 62 is adhesively secured to the polymeric layer 60. In some cases, while not shown, the conductive member 62 may be partially embedded within the polymeric layer 60.

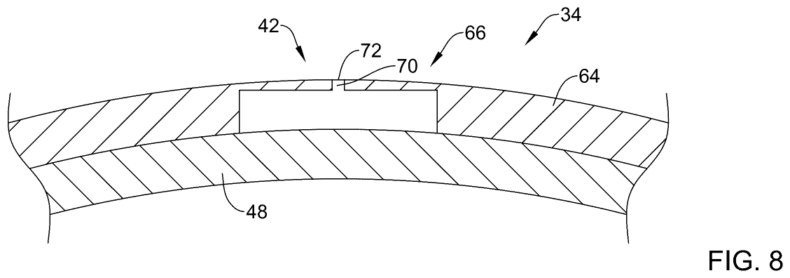

FIG. 8 provides another example of the inflatable balloon 34 having a dual wall construction, with the wall 48 now forming an inner wall. An insulative layer 64 forms at least a partial outer wall. The insulative layer 64 may be formed of any suitable polymer that is electrically insulating. The electrocautery electrode 42 includes a conductive member 66 that is partially embedded within the insulative layer 64. In some cases, as shown, the conductive member 66 may include a base portion 68 that is embedded within the insulative layer 64 and an extension portion 70 that extends outwardly from the base portion 68. The extension portion 70 extends through the insulative layer 64 and an end surface 72 extends free of the insulative layer 64 and provides an electrode surface. The conductive member 66 may be made from any desired electrically conductive material such as a metal. In some cases, the conductive member 66 may be made of, or coated with, gold. The insulative layer 64 may be formed of any suitable electrically insulating polymer that is biocompatible.

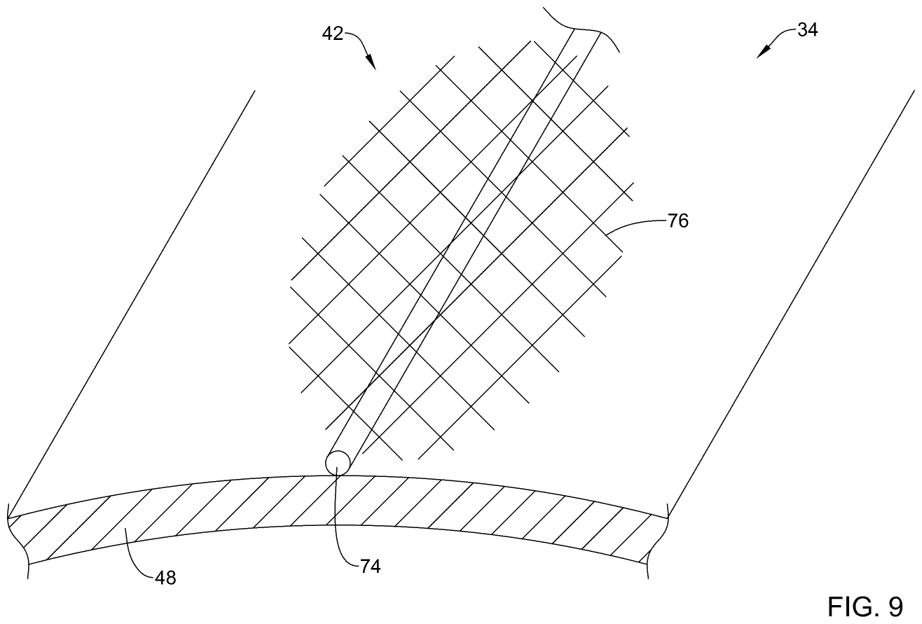

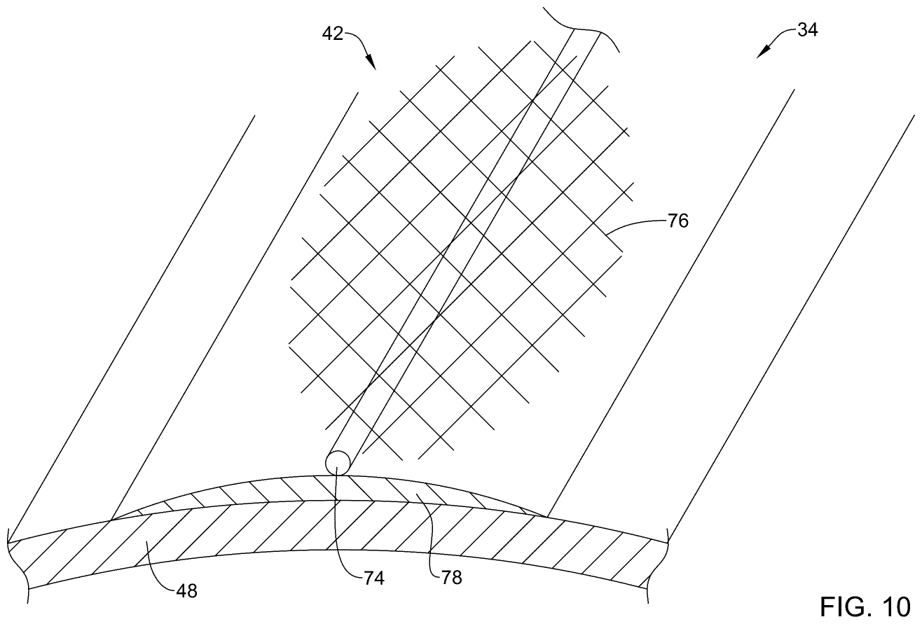

FIG. 9 provides another example of the inflatable balloon 34 having a single wall construction, with a conductive member 74 secured relative to the inflatable balloon 34 via a mesh. A conductive member 74 sits atop the wall 48. A mesh 76 is secured to the wall 48 and holds the conductive member 74 in place relative to the wall 48. Because the mesh 76 is porous, RF energy emitted by the conductive member 74 is able to pass through the mesh 76 and reach the valve leaflets 120 (or the valve leaflets 14). The mesh 76 may be adhesively secured to the wall 48. In some cases, the mesh 76 may be partially embedded into the wall 48. The mesh 76 may be formed of any suitable electrically insulating material such as a polymer. FIG. 10 is similar, but shows the addition of an electrically insulative layer 78 between the wall 48 and the mesh 76. The electrically insulative layer 78 may be formed of any suitable biocompatible electrically insulative polymer. Examples of suitable biocompatible electrically insulative polymers include polyimides, liquid crystal polymers (LCP), polyamides, silicone and various thermopolymers. Additional examples include fluoropolymers such as PTFE (polytetrafluoroethylene), FEP (fluorinated ethylene propylene), PVDF (polyvinylidene fluoride) and PVDF-HFP (poly(vinylidene fluoride-hexafluoropropylene). Some of these materials may be used as tubes or heat shrinks. Some of these materials may be cast or molded, or may be sprayed.

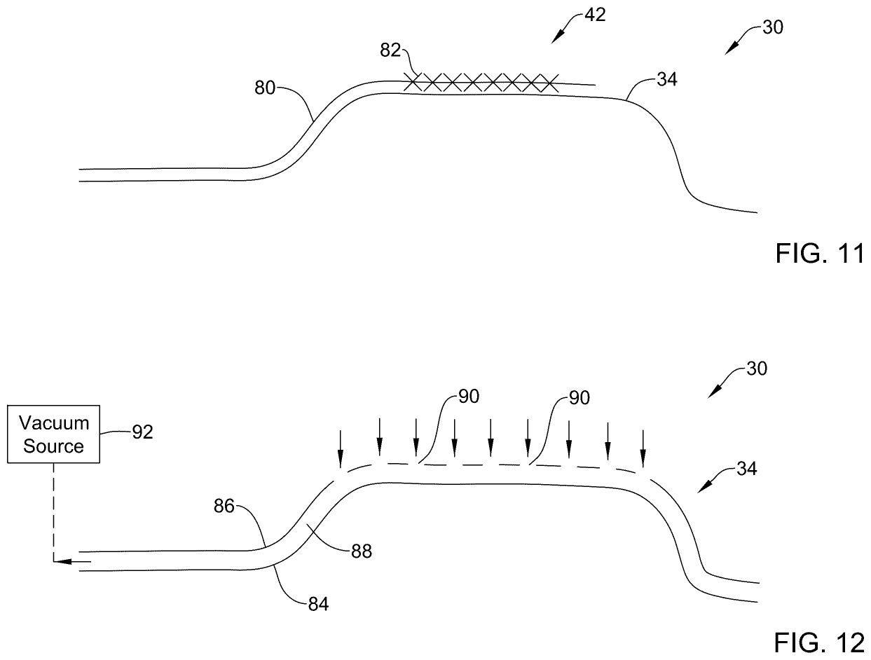

In some instances, there may be a desire to be able to capture and remove any excised valve leaflet portions (regardless of whether the excised valve leaflet portions are part of the valve leaflets 120 or the valve leaflets 14). In some cases, an outer surface of the inflatable balloon 34 may be adapted to help capture and subsequently remove any excised valve leaflet portions. FIGS. 11 through 14 provide illustrative but non-limiting examples of balloon constructions that can facilitate capturing and removing any excised valve leaflet portions. In FIG. 11, an electrical conductor 80 may be seen extending along an outer surface of the inflatable balloon 34. A distal portion of the electrical conductor 80 forms the electrocautery electrode 42. The medical device 30 includes a layer 82 that is adapted to contact and adhere to any excised valve leaflet portions. In some cases, the layer 82 may include an adhesive material that by itself may adhere to any excised valve leaflet portions. The adhesive material may be applied to the inflatable balloon 34 via a post-balloon molding deposition process. In some cases, a temporary shield layer (not shown) may be disposed over the adhesive material. The temporary shield layer may dissolve or delaminate from the adhesive material once the temporary shield layer is exposed to water, blood or saline. In some cases, the surface 82 may include part of a hook and loop attachment mechanism (commonly known as VELCRO®) that can adhere to any excised valve leaflet portions. In some cases, part of a hook and loop attachment mechanism may be secured to the inflatable balloon 34 using a thermo-polymeric bond, an adhesive or sutures. Part of a hook and loop attachment mechanism may be post-molded onto the inflatable balloon 34, for example.

FIG. 12 shows a medical device 30 in which the inflatable balloon 34 has an inner layer 84 and an outer layer 86 that is spaced from the inner layer 84 in order to form a space 88 that is defined between the inner layer 84 and the outer layer 86. In some cases, the outer layer 86 includes a number of pores or openings 90 that provide fluid communication between the space 88 and the environment external to the inflatable balloon 34. In some cases, a source of vacuum 92 may be fluidly coupled to the space 88 between the inner layer 84 and the outer layer 86. By coupling the source of vacuum 92 (commonly available in many procedure rooms) to the space 88 between the inner layer 84 and the outer layer 86, any excised valve leaflet portion that is close to the inflatable balloon 34 will be sucked into contact with the outer layer 86. By deflating the inflatable balloon 34 while still applying vacuum, any excised valve leaflet portion may be easily removed from the patient. While not shown for clarity, the inflatable balloon 34 includes the electrocautery electrode 42.

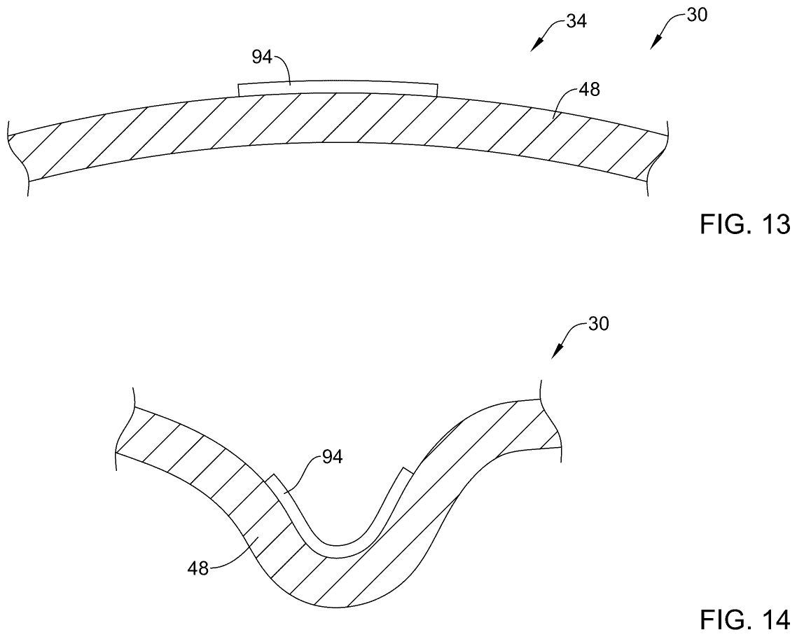

FIG. 13 shows a medical device 30 in which an excised valve leaflet portion 94 is adjacent to the inflatable balloon 34. The inflatable balloon 34 is shown in an inflated configuration in FIG. 13. In FIG. 14, the inflatable balloon 34 is shown partially deflated, such as the inflatable balloon 34 would be after beginning to deflate the inflatable balloon 34 but before the inflatable balloon 34 is fully deflated. As the inflatable balloon 34 deflates, and thus folds in on itself, in some cases the excised valve leaflet portion 94 may also get folded into the deflating inflatable balloon 34. This can hold the excised valve leaflet portion 94 relative to the deflating inflatable balloon 34 sufficiently to allow the excised valve leaflet portion 94 to be removed when the medical device 30 is removed from the patient. In some cases, the folding characteristics of the inflatable balloon 34 may be sufficient to hold the excised valve leaflet portion 94 in position relative to the deflating inflatable balloon 34. In some cases, additional measures may be included in the medical device 30, such as the layer 82 shown in FIG. 11 or the vacuum mechanism shown in FIG. 12.

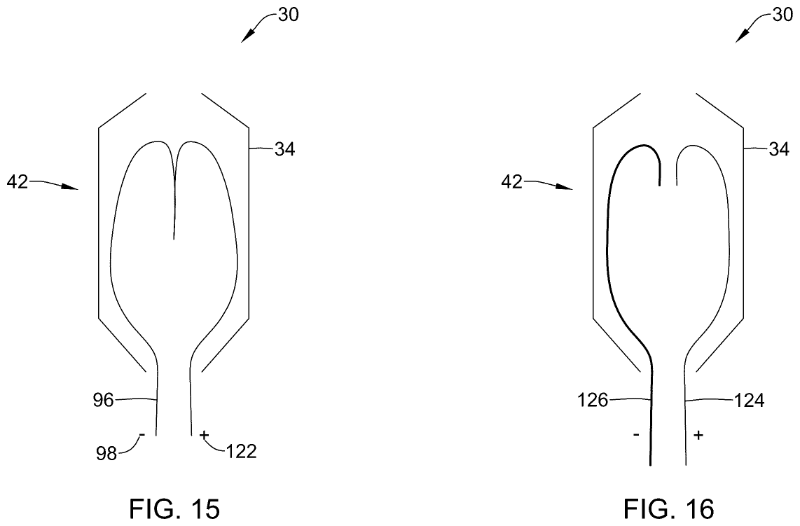

FIG. 15 is a schematic view of the medical device 30, showing a mono-polar arrangement for the electrocautery electrode 42. An electrode 96 extends from a ground 98 to a positive (+) power supply 122. In contrast, FIG. 16 is a schematic view of the medical device 30, showing a bi-polar arrangement for the electrocautery electrode 42. As shown, a positive (+) electrode 124 extends across half of the inflatable balloon 34 and a negative (−) electrode 126 extends across the other half of the inflatable balloon 34, but does not contact the positive (+) electrode 124. Having a bi-polar arrangement for the electrocautery electrode 42 means that another electrical contact elsewhere on the patient is not needed.

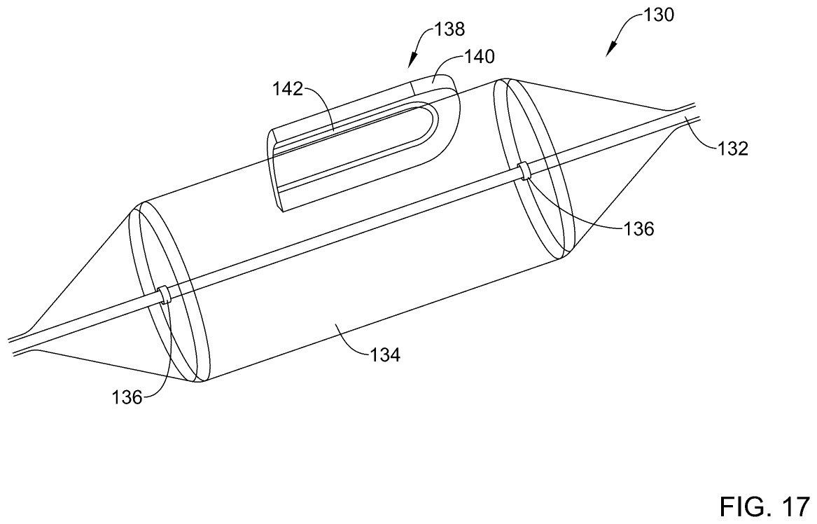

In some cases, an ancillary device may be used in combination with the medical device 30, or may be included as part of the medical device 30. FIG. 17 is a schematic view of a medical device 130 that includes an inflatable balloon 134 on an elongate shaft 132. The elongate shaft 132 extends through the inflatable balloon 134, and includes several radiopaque marker bands 136 that may be used to better position the medical device 130 fluoroscopically. The medical device 130 includes an ancillary device 138. The ancillary device 138 includes an electrocautery electrode 142. During use, the ancillary device 138 (which may include an elongate shaft (not shown) that allows advancement and placement of the ancillary device 138) may be placed between the valve leaflets 120 (or the valve leaflets 14) and either the framework 110 of the heart valve implant 100 or the native heart valve. The inflatable balloon 134 may be extended through the valve (native or artificial) and inflated to push the valve leaflets into contact with the electrocautery electrode 142 on the ancillary device 138. In some cases, placing the electrocautery electrode 142 on the ancillary device 138 may permit use of a lower power level because less energy may be lost to blood. The ancillary device 138 may also help in preventing the electrocautery electrode 142 from contacting the metal framework 110 of the heart valve implant 100.

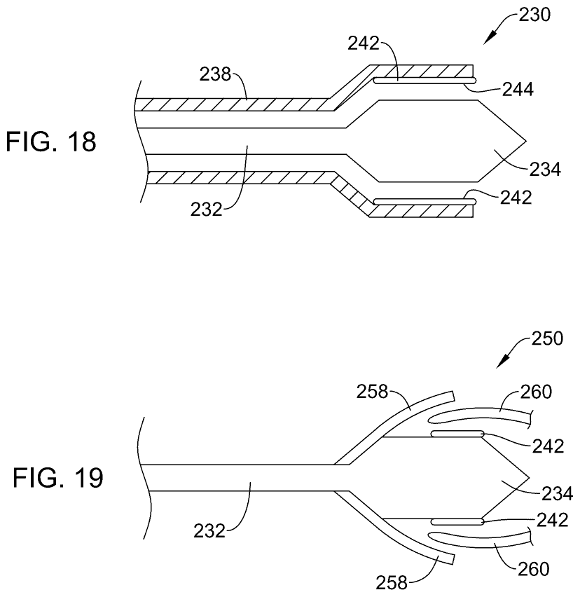

FIG. 18 is a schematic view of an illustrative medical device 230 that includes an elongate shaft 232 bearing an inflatable balloon 234. An ancillary device 238 extends over the inflatable balloon 234 and includes one or more electrocautery electrodes 242 on an inner surface of the inflatable balloon 234. In use, the ancillary device 238 is advanced distally over the inflatable balloon 234. A space 244 is defined between the inflatable balloon 234 and the electrocautery electrode(s) 242 that is adapted to allow a valve leaflet to extend into. Inflating the inflatable balloon 234 forces the valve leaflet against the electrocautery electrode(s) 242 such that a portion of the valve leaflet may be excised. The ancillary device 238 may be a separate element from the medical device 230, or may be included as part of the medical device 230 as long as the ancillary device 238 and the inflatable balloon 234 can be separately translated.

FIG. 19 is a schematic view of an illustrative medical device 250 that includes an elongate shaft 232 bearing an inflatable balloon 234. The inflatable balloon 234 includes one or more electrocautery electrode(s) 242 on an outer surface of the inflatable balloon 234. The medical device 250 includes protective devices 258 that extend over the inflatable balloon 234 and define a space into which valve leaflets 260 may extend. The protective devices 258 shield surrounding anatomy from RF energy applied via the one or more electrocautery electrode(s) 242.

The materials that can be used for the various components of the devices and various elements thereof disclosed herein may include those commonly associated with medical devices. In some instances, the medical devices, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), clastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfonc, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the medical devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the medical devices and/or other elements disclosed herein. For example, the medical devices, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the medical devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical device adapted for excising a valve leaflet portion from a heart valve, the medical device comprising:
an elongate shaft including a distal region;
an inflatable balloon secured to the distal region and adapted to extend through the heart valve, the inflatable balloon having an outer surface and including a deflated configuration and an inflated configuration; and
an electrocautery electrode disposed relative to the inflatable balloon such that the electrocautery electrode is positioned to excise the valve leaflet portion when RF energy is applied to the electrocautery electrode and the inflatable balloon is in the inflated configuration.

2. The medical device of claim 1, wherein the heart valve comprises an artificial heart valve.

3. The medical device of claim 2, wherein the valve leaflet comprises an artificial valve leaflet.

4. The medical device of claim 1, wherein the outer surface of the inflatable balloon is adapted to capture the excised valve leaflet portion.

5. The medical device of claim 4, further comprising a layer disposed on the outer surface of the inflatable balloon that is to adapted capture the excised valve leaflet portion.

6. The medical device of claim 4, wherein:
the inflatable balloon includes an inner balloon wall and an outer balloon wall defining a space therebetween, the outer balloon wall defining the outer surface;
the outer balloon wall comprising a plurality of pores; and
a source of vacuum fluidly coupled with the space defined between the inner balloon wall and the outer balloon wall.

7. The medical device of claim 4, wherein the outer surface of the inflatable balloon is adapted to fold over the excised valve leaflet portion when the inflatable balloon is deflated.

8. The medical device of claim 1, wherein the electrocautery electrode is disposed on the outer surface of the inflatable balloon.

9. The medical device of claim 8, further comprising an insulative layer disposed on the outer surface of the inflatable balloon.

10. The medical device of claim 1, further comprising an ancillary device adapted to extend at least partially around the inflatable balloon.

11. The medical device of claim 10, wherein the electrocautery electrode is disposed on the ancillary device.

12. The medical device of claim 1, wherein the electrocautery electrode comprises a mono-polar loop electrode.

13. The medical device of claim 1, wherein the electrocautery electrode comprises a bi-polar loop electrode.

14. A medical device adapted for excising a portion of an aortic valve leaflet from an aortic valve, the medical device comprising:
an elongate shaft including a distal region;
an inflatable balloon secured to the distal region and adapted to extend through the aortic valve, the inflatable balloon having an outer surface and including a deflated configuration and an inflated configuration;
an ancillary device adapted to extend at least partially over the inflatable balloon; and
an electrocautery electrode disposed between the inflatable balloon and the ancillary device such that the electrocautery electrode is positioned to excise the portion of the aortic valve leaflet when energy is applied to the electrocautery electrode and the inflatable balloon is in the inflated configuration.

15. The medical device of claim 14, wherein the aortic valve comprises a previously implanted artificial aortic valve.

16. The medical device of claim 14, wherein the outer surface of the inflatable balloon is adapted to capture a portion of the aortic valve leaflet excised by the electrocautery electrode.

17. A medical device adapted for excising material from a heart valve, the medical device comprising:
an elongate shaft including a distal region;
an inflatable balloon secured to the distal region and adapted to extend through the heart valve, the inflatable balloon having an outer surface and including a deflated configuration and an inflated configuration; and
an electrode disposed relative to the inflatable balloon such that the electrode is positioned to excise the material when energy is applied to the electrode and the inflatable balloon is in the inflated configuration;
wherein the outer surface of the inflatable balloon is adapted to capture the material excised by the electrode.

18. The medical device of claim 17, further comprising an adhesive layer disposed on at least a portion of the outer surface of the inflatable balloon.

19. The medical device of claim 17, wherein:
the inflatable balloon includes an inner balloon wall and an outer balloon wall defining a space therebetween, the outer balloon wall defining the outer surface;
the outer balloon wall comprising a plurality of pores; and
a source of vacuum fluidly coupled with the space defined between the inner balloon wall and the outer balloon wall.

20. The medical device of claim 17, wherein the outer surface of the inflatable balloon is adapted to fold over the excised portion of the valve leaflet when the inflatable balloon is deflated.
